(19) United States
(12) Patent Application Publication
(10) Pub. No.: US 2003/0065263 A1
(43) Pub. Date: Apr. 3, 2003

(54) Ultrasonic probe device with rapid attachment and detachment means having a line contact collet

(75) Inventors: Bradley A. Hare, Chelmsford, MA (US); Robert A. Rabiner, North Reading, MA (US); Kevin J. Ranucci, North Attleboro, MA (US); Rebecca I. Marciante, North Reading, MA (US); Mark J. Varady, Marlborough, MA (US); Roy M. Robertson, Ipswich, MA (US); Janniah S. Prasad, Norwalk, CT (US); Scott A. Talbot, North Andover, MA (US)

Correspondence Address: PALMER & DODGE, LLP, RICHARD B. SMITH, 111 HUNTINGTON AVENUE, BOSTON, MA, 02199, US

(73) Assignee: Omnisonics Medical Technologies, Inc.

(21) Appl. No.: 10/268,843

(22) Filed: Oct. 10, 2002

Related U.S. Application Data
(63) Continuation-in-part of application No. 09/975,725, filed on Oct. 11, 2001.
Application No. 09/975,725 is a continuation-in-part of application No. 09/625,803, filed on Jul. 26, 2000.
(60) Provisional application No. 60/157,824, filed on Oct. 5, 1999.

(51) Int. Cl. A61B 8/00

(52) U.S. Cl. 600/439

(57) ABSTRACT

An ultrasonic medical device comprising an ultrasonic probe and a collet assembly for probe attachment and detachment, and a method of removing occlusions in blood vessels using the ultrasonic medical device. The probe detachability allows insertion, manipulation and withdrawal independently of a device body. The collet assembly comprises a compression clamp capable of releasably receiving the probe, and a compression housing that initiates a minimal area “line-contact” between the collet assembly segments upon engagement. A line-contact lip ensures consistent and repeatable contact between the compression clamp and the compression housing at a predetermined location to provide a consistent closing force on the probe for any selected tightening torque. The line-contact between the collet assembly segments provides efficient ultrasonic energy transfer from an ultrasonic energy source to the probe thereby increasing the probe efficiency during tissue ablation.

### RELATED APPLICATIONS

This application is a continuation in part of U.S. application Ser. No. 09/975,725 filed on Oct. 11, 2001, which is a continuation in part of U.S. application No. 09/625,803 filed on Jul. 26, 2000, which claims priority to U.S. Provisional Application No. 60/157,824 filed on Oct. 5, 1999, the entirety of which is hereby incorporated by reference.

### FIELD OF THE INVENTION

The present invention relates generally to a rapid attachment and detachment system for an ultrasonic probe used for tissue ablation capable of efficiently transferring ultrasonic energy from the ultrasonic energy source to the probe with minimal energy loss. Specifically, the present invention relates to an attachment and detachment system that is capable of retaining the ultrasonic probe by maintaining a minimal area of contact or “line-contact” between the probe attachment segment and the ultrasonic probe so as to provide optimal energy transfer from the ultrasonic energy source in the handle segment of the probe, thereby increasing probe efficiency during tissue ablation.

### BACKGROUND OF THE INVENTION

Vascular occlusions (clots or thrombi and occlusional deposits, such as calcium, fatty deposits, or plaque) result in the restriction or blockage of blood flow in the vessels in which they occur. Occlusions result in oxygen deprivation (“ischemia”) of tissues supplied by these blood vessels. Prolonged ischemia results in permanent damage of tissue that can lead to myocardial infarction, stroke, or death. Targets for occlusion include coronary arteries, peripheral arteries and other blood vessels. The disruption of an occlusion or thrombolysis can be effected by pharmacological agents and/or mechanical means.

Ultrasonic probes are devices which use ultrasonic energy to fragment body tissue (see, e.g., U.S. Pat. No. 5,112,300; U.S. Pat. No. 5,180,363; U.S. Pat. No. 4,989,583; U.S. Pat. No. 4,931,047; U.S. Pat. No. 4,922,902; and U.S. Pat. No. 3,805,787) and have been used in many surgical procedures. The use of ultrasonic energy has been proposed both to mechanically disrupt clots, and to enhance the intravascular delivery of drugs to clot formations (see, e.g., U.S. Pat. No. 5,725,494; U.S. Pat. No. 5,728,062; and U.S. Pat. No. 5,735,811). Ultrasonic devices used for vascular treatments typically comprise an extra-corporeal transducer coupled to a solid metal wire that is attached to a plurality of wires at the distal end that is then threaded through the blood vessel and placed in contact with the occlusion (see, e.g., U.S. Pat. No. 5,269,297). In some cases, the transducer is delivered to the site of the clot, the transducer comprising a bendable plate (see, U.S. Pat. No. 5,931,805).

The ultrasonic energy produced by an ultrasonic probe is in the form of very intense, high frequency sound vibrations that result in powerful chemical and physical reactions in the water molecules within a body tissue or surrounding fluids in proximity to the probe. These reactions ultimately result in a process called “cavitation,” which can be thought of as a form of cold (i.e., non-thermal) boiling of the water in the body tissue, such that microscopic bubbles are rapidly created and destroyed in the water creating cavities in their wake. As surrounding water molecules rush in to fill the cavity created by collapsed bubbles, they collide with each other with great force. This process is called cavitation and results in shock waves running outward from the collapsed bubbles which can fragment or ablate material such as surrounding tissue in the vicinity of the probe.

Some ultrasonic probes include a mechanism for irrigating an area where the ultrasonic treatment is being performed (e.g., a body cavity or lumen) to wash tissue debris from the area. Mechanisms used for irrigation or aspiration described in the art are generally structured such that they increase the overall cross-sectional profile of the probe, by including inner and outer concentric lumens within the probe to provide irrigation and aspiration channels for removal of particulate matter. In addition to making the probe more invasive, prior art probes also maintain a strict orientation of the aspiration and the irrigation mechanism, such that the inner and outer lumens for irrigation and aspiration remain in a fixed position relative to one another, which is generally closely adjacent the area of treatment. Thus, the irrigation lumen does not extend beyond the suction lumen (i.e., there is no movement of the lumens relative to one another) and any aspiration is limited to picking up fluid and/or tissue remnants within the defined distance between the two lumens.

Another drawback of existing ultrasonic medical probes is that they typically remove tissue relatively slowly in comparison to instruments that excise tissue by mechanical cutting. Part of the reason for this is that existing ultrasonic devices rely on a longitudinal vibration of the tip of the probe for their tissue-disrupting effects. Because the tip of the probe is vibrated in a direction in line with the longitudinal axis of the probe, a tissue-destroying effect is only generated at the tip of the probe. One solution that has been proposed is to vibrate the tip of the probe in a direction other than perpendicular to the longitudinal axis of the probe, in addition to vibrating the tip in the longitudinal direction. It is proposed that such motions will supplement the main point of tissue destruction, which is at the probe tip, since efficiency is determined by surface area of the probe tip. For example, U.S. Pat. No. 4,961,424 to Kubota, et al. discloses an ultrasonic treatment device that produces both a primary longitudinal motion, and a supplementary lateral motion of the probe tip to increase the tissue disrupting efficiency. The Kubota, et al. device, however, still relies primarily on the tip of the probe to act as a working surface. The ancillary lateral motion of the probe is intended to provide an incremental efficiency for the device operation. Thus, while destruction of tissue in proximity to the tip of the probe is more efficient, tissue destruction is still predominantly limited to the area in the immediate vicinity at the tip of the probe. The Kubota, et al. device is therefore limited in its ability to ablate tissue within inner surfaces of cylindrical blood vessels, for example, in vascular occlusions. U.S. Pat. No. 4,504,264 to Kelman discloses an ultrasonic treatment device containing a probe that is capable of longitudinal vibrations and lateral oscillation. The Kelman device is intended to improve the efficiency of ultrasonic tissue removal by providing a dual function of a fragmentation and a cutting device. Tissue fragmentation is caused primarily by oscillating the tip of the probe in addition to relying on longitudinal vibrations of the probe. Tissue fragmentation is caused primarily at the tip of the device, while the oscillatory motion can be employed by the surgeon to cut tissue, thereby increasing efficiency of surgical procedures. The prior art devices also require complex instrument design that require incorporation of a plurality of electrodes, ultrasound frequency generating elements, switches or voltage controllers.

The longitudinal probe vibration required for tissue ablation in prior art devices necessitates the probe lengths to be relatively short, since use of long probes result in a substantial loss of ultrasonic energy at the probe tip due to thermal dissipation and undesirable horizontal vibration that interferes with the required longitudinal vibration.

Effecting ultrasonic transmission through a plurality of flexible thin wires has been found impracticable because: (1) relatively high power (˜25 watts) is required to deliver sufficient energy to the probe tip; and (2) such thin wires tend to perform buckling vibrations, resulting in almost the entire ultrasonic power introduced in the probe is dissipated during its passage to the probe tip. Such limitations have precluded the use of ultrasonic tissue ablation devices in surgical procedures wherein access to vascular occlusion requires traversing an anatomically lengthy or sharply curved path along tubular vessels. Furthermore, the relatively high-energy requirement for such devices causes probe heating that can cause fibrin to re-clot blood within the occluded vessel (thermally induced re-occlusion). The elevation in probe temperature is not just limited to probe tip, but also occurs at points wherein the narrow diameter wire probes have to bend to conform to the shape of the blood vessel, thereby limiting causing probe damage and limiting its reuse.

A single thick wire probe on the other hand, cannot negotiate the anatomical curves of tubular arterial and venous vessels due to its inflexibility, and could cause damage to the interior wall of such vessels. Currently, such exchange procedures are not possible because ultrasonic probes used in endovascular procedures are permanently attached to the transducer energy source or a probe handle coupled to such source, such as for example, by welding, thereby precluding probe detachment. Moreover, since probe vibration in such devices in a longitudinal mode, i.e. along the probe longitudinal axis, a proximal contact with the transducer or the probe handle segment connect is essential to prevent a “hammering” effect that can result in probe damage.

In prior art collets for ultrasonic probes, the inner surface of the collet housing mates with the surface of the collet base in an unpredictable and inconsistent manner because of the difficulty in matching the angle of taper on the housing and the base. Even though the collets can be produced on highly accurate machines (i.e., computer controlled lathes, numerically controlled screw machines, etc.), the machine tolerances still create collets that vary within a few thousandths of an inch, which is not an unusual tolerance. Thus, the position of the line contact is inconsistent from collet to collet and can only be located at either the front end or the back end of the collet and cannot be located in the middle. For the same tightening torque, the actual closing force can vary over a wide range from collet to collet.

Thus, there is a need in the art for an improvement over the prior art collets to provide a consistent closing force on the wire for any selected tightening torque. There is also a need in the art for a coupling mechanism for an ultrasonic probe to be releasably coupled to an ultrasonic energy source in a manner to minimize undesirable vibrations that can cause probe damage, and enable efficient transfer of ultrasound energy from an energy source to the ultrasonic probe to maximize its tissue ablation capability.

### SUMMARY OF THE INVENTION

The present invention is a rapidly attachable and detachable or “quick attachment-detachment” system (referred to herein as “QAD”) for an elongated catheter probe of an ultrasonic tissue ablation device, wherein the probe is capable of ultrasonically vibrating substantially in a direction transverse to the probe longitudinal axis for coupling or decoupling it from the ultrasonic energy source. Manipulation and positioning of the probe within narrow body vessels, such as for example vascular arteries, can be accomplished without being limited by the relatively bulky energy source. Specifically, the present invention relates to a coupling mechanism or “collet assembly” that enables rapid attachment and detachment of the ultrasonic probe of the device in a manner so as to maintain a minimal area of contact between the collet assembly segments. This, in turn, enables optimal energy transfer from the ultrasonic energy source of the device to the ultrasonic probe, thereby increasing probe efficiency during tissue ablation. This objective of the present invention is accomplished by providing an attachment and detachment collet assembly that is capable of retaining the ultrasonic probe by maintaining a minimal area of contact or “line-contact” between the probe attachment segment in the collet assembly and the ultrasonic probe.

In one aspect, the present invention provides a probe attachment-detachment system or “collet assembly” that is capable of detachably restraining an ultrasonic catheter probe by maintaining a minimal area of contact, thereby transferring ultrasonic energy from a source to the probe in an optimal and efficient manner.

In another aspect, the present invention provides a guide wire probe assembly with the aforementioned collet that enables intravascular ultrasonic tissue ablation in long and narrow-diameter blood vessels.

Additionally, the present invention provides a method of removing an occlusion in a blood vessel using an ultrasonic device having a quick attachment and detachment line-contact collet assembly.

Additional aspects and features of the present invention will become apparent from the following description, wherein the preferred embodiments are set forth in detail in conjunction with accompanying figures.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows the collet assembly of the present invention that is coupled to a hand piece of an ultrasonic tissue ablation device housing an energy source and an ultrasonic probe that is removably attached to the collet assembly.

FIG. 2A and FIG. 2B illustrate the prior art problem of random variability in the line contact between the compression housing and the compression clamp. FIG. 2A illustrates an unevenly matched contact mating toward a back end of the compression housing while FIG. 2B illustrates an unevenly matched contact mating toward a front end of the compression housing.

FIG. 3 shows a cross-sectional view of the line-contact collet assembly of the present invention.

FIGS. 4A, 4B and 4C show the line-contact collet assembly of the present invention in an attached mode (FIG. 4A), in a detached mode (FIG. 4B), and in a cross-section in the detached mode (FIG. 4C).

FIG. 5 shows the compression housing component and highlights the line-contact lip at the terminal end of the housing.

FIG. 6A and FIG. 6B show cross-sectional views of the line-contact collet assembly of the present invention prior to attachment to an ultrasonic probe (FIG. 6A) and after attachment to an ultrasonic probe (FIG. 6B).

FIG. 7 is a general view of the elongated flexible ultrasonic probe of the present invention.

FIG. 8 shows the threaded horn component of the QAD collet-horn assembly.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION OF THE INVENTION

The following terms and definitions are used herein:

“Cavitation” as used herein refers to shock waves produced by ultrasonic vibration, wherein the vibration creates a plurality of microscopic bubbles which rapidly collapse, resulting in molecular collision by water molecules which collide with force thereby producing the shock waves.

“Fenestration” as used herein refers to an aperture, window, opening, hole, or space.

“Node” as used herein refers to a region of minimum energy emitted by an ultrasonic probe at or proximal to a specific location along the longitudinal axis probe.

“Anti-node” as used herein refers to a region of maximum energy emitted by an ultrasonic probe at or proximal to a specific location along the longitudinal axis probe.

“Probe” as used herein refers to a device capable of being adapted to an ultrasonic generator means, which is capable of propagating the energy emitted by the ultrasonic generator means along its length, resolving this energy into effective cavitational energy at a specific resonance (defined by a plurality of nodes and anti-nodes at a pre-determined locations (defined as an “active area” of the probe)) and is capable of acoustic impedance transformation of ultrasound energy to mechanical energy.

“Sheath” as used herein refers to a device for covering, encasing, or shielding in whole or in part, a probe or portion thereof connected to an ultrasonic generation means.

“Transverse” as used herein refers to vibration of a probe not parallel to the longitudinal axis of a probe. A “transverse wave” as used herein is a wave propagated along an ultrasonic probe in which the direction of the disturbance at each point of the medium is perpendicular to the wave vector.

“Tuning” as used herein refers to a process of adjusting the frequency of the ultrasonic generator means to select a frequency that establishes a standing wave along the length of the probe.

The present invention provides an ultrasonic medical device operating in a transverse mode for removing a vascular occlusion by causing fragmentation of occlusion materials such as tissue. Because the device is minimally invasive, flexible and articulable, it can be inserted into narrow, tortuous blood vessels without risking damage to those vessels. Transverse vibration of the probe in such a device generates multiple anti-nodes of cavitation energy along the longitudinal axis of the probe, which are resolved into cavitational anti-nodes emanating radially from these anti-nodes at specific points along the probe. The occlusion tissue is fragmented to debris approximately of sub-micron sizes, and the transverse vibration generates a retrograde flow of debris that carries the debris away from the probe tip.

The transverse mode of vibration of the ultrasonic probe according to the invention differs from the axial (or longitudinal) mode of vibration that is conventional in the prior art. Rather than vibrating in the axial direction, the probe vibrates substantially in a direction transverse (perpendicular) to the axial direction. As a consequence of the transverse vibration of the probe, the tissue-destroying effects of the device are not limited to those regions of a tissue coming into contact with the tip of the probe. Rather, as the active portion of the probe is positioned in proximity to an occlusion or other blockage of a blood vessel, the tissue is removed in all areas adjacent to the multiplicity of energy anti-nodes that are produced along the entire length of the probe, typically in a region having a radius of up to about 6 mm around the probe.

By allowing transverse vibrations, fragmentation of large areas of tissue spanning the entire length of the active portion of the probe due to generation of multiple cavitational anti-nodes along the probe length perpendicular to the probe axis occurs. Since substantially larger affected areas within an occluded blood vessel can be denuded of the occluded tissue in a short time, actual treatment time using the transverse mode ultrasonic medical device according to the invention is greatly reduced as compared to methods using prior art probes that primarily utilize longitudinal vibration (along probe axis) for tissue ablation. A distinguishing feature of the present invention is the ability to utilize probes of extremely small diameter (about 0.025″ and smaller) compared to prior art probes without loss of efficiency, since the tissue fragmentation process is not dependent on area of the probe tip (distal end). Highly flexible probes can therefore, be designed to mimic device shapes that enable facile insertion into highly occluded or extremely narrow interstices within blood vessels. Another advantage provided by the present invention is its ability to rapidly remove occlusion tissue from large areas within cylindrical or tubular surfaces such as arteries and arterial valves or selected areas within the tubular walls, which is not possible by previously disclosed devices that rely on the longitudinal vibrating probe tip for effecting tissue fragmentation.

An ultrasonic probe functioning in a transverse mode facilitates efficient operation of narrow-diameter probes for rapid tissue ablation. Transversely vibrating ultrasonic probes for tissue ablation are described in the assignee's co-pending patent applications U.S. Ser. Nos. 09/975,725; 09/618,352; and 09/917,471, the entirety of those applications are hereby incorporated by reference. These co-pending patent applications describe the design parameters for such a probe its use in ultrasonic devices for tissue ablation. An ultrasonic probe vibrating in a transverse mode for removal of occlusions in blood vessels has been disclosed in assignee's co-pending patent application Ser. No. 09/776,015, the entirety of which is hereby incorporated as reference. This co-pending patent application discloses an ultrasonic device in which a transducer is connected to a probe with a flexible tip capable of vibrating in a direction transverse to the probe longitudinal axis. With such a probe a situation may arise where it will be desirable to utilize an elongated probe resembling a catheter guide-wire probe to make possible exchange procedures often used in angioplasty.

The number of anti-nodes occurring along the axial length of the probe is modulated by changing the frequency of energy supplied by the ultrasonic generator. The exact frequency, however, is not critical and a ultrasonic generator run at, for example, 20 kHz is generally sufficient to create an effective number of tissue destroying anti-nodes along the axial length of the probe. In addition, as will be appreciated by those skilled in the art, it is possible to adjust the dimensions of the probe, including diameter, length, and distance to the ultrasonic energy generator, in order to affect the number and spacing of anti-nodes along the probe. The present invention allows the use of ultrasonic energy to be applied to tissue selectively, because the probe transmits energy across a frequency range of from about 20 kHz through about 80 kHz. The amount of ultrasonic energy to be applied to a particular treatment site is a function of the amplitude and frequency of vibration of the probe. In general, the amplitude or throw rate of the energy is in the range of 25 microns to 250 microns, and the frequency in the range of 20,000 to 80,000 Hertz (20-80 kHz). In the currently preferred embodiment, the frequency of ultrasonic energy is from 20,000 Hertz to 35,000 Hertz (20-35 kHz). Frequencies in this range are specifically destructive of hydrated (water-laden) tissues and vascular occlusive material, while substantially ineffective toward high-collagen connective tissue, or other fibrous tissues such as, for example, vascular tissues, skin or muscle tissues.

In a preferred embodiment, the ultrasonic medical device of the present invention, comprises an ultrasonic generator that is mechanically coupled to a probe having a proximal and distal end that is capable of oscillating in a direction transverse to its longitudinal axis. Alternatively, a magneto-strictive generator may be used for generation of ultrasound energy. The preferred generator is a piezoelectric transducer that is mechanically coupled to the probe to enable transfer of ultrasonic excitation energy and cause the probe to oscillate in a transverse direction relative to its longitudinal axis. The device is designed to have a small cross-sectional profile, which also allows the probe to flex along its length, thereby allowing it to be used in a minimally invasive manner. Transverse oscillation of the probe generates a plurality of cavitation anti-nodes along the longitudinal axis of the member, thereby efficiently destroying the occlusion. A significant feature of the invention is the retrograde movement of debris, e.g., away from the tip of the probe i.e. backwards up along the shaft of the probe that results from the transversely generated energy. The amount of cavitation energy to be applied to a particular site requiring treatment is a function of the amplitude and frequency of vibration of the probe, as well as the longitudinal length of the probe tip, the proximity of the tip to a tissue, and the degree to which the probe tip is exposed to the tissues.

A distinguishing feature of the present invention is the ability to utilize probes of extremely small diameter (narrow diameter probes) compared to previously disclosed devices (large diameter probes) without loss of efficiency or efficacy, since the tissue fragmentation process is not dependent on area of the probe tip (distal end). Highly flexible probes can therefore be obtained to mimic device shapes that enable facile insertion into highly occluded or extremely narrow interstices without resulting in breakage of the probe or puncture or damage of the tissue or body cavity while ensuring optimal results.

A second distinguishing feature of the small diameter probes of the invention is that the probe diameter is approximately the same over their entire length, that is, the active tip segment (distal end) and the rear segment (proximal end) of the probes are approximately similar in diameter. In a preferred embodiment, the probe diameter at the proximal end is about 0.025 inches and the probe diameter at the distal end is about 0.015 inches, so the probe is adaptable to standard vascular introducers. Since the rear segment (proximal end) of the probes have no non-cylindrical shape or “bulk”, catheters and guides can be introduced over the ends of the elongated wire probes of the invention, thereby allowing their use in standard configuration endovascular procedures.

The ultrasonic device of the invention comprises a longitudinal resonator such as for example, a Mason (Langevin) horn that is in intimate contact with an elongated catheter wire probe through a collet assembly. The horn assembly is in turn, connected to an ultrasound energy source. Upon device activation, ultrasonic energy from the source is transmitted to the horn assembly wherein it is amplified by the horn and in turn, transmitted to the probe thorough the collet assembly. Transverse vibrational modes along the longitudinal axis of the probe that are coupled to the horn resonance will be excited upon the delivery of ultrasonic energy to the probe.

The coupling between the elongated probe and the horn is adjusted so as to present a relatively large impedance mismatch, and be located at an anti-node of the horn. Longitudinal waves impinging on the coupling interface are either reflected back into the horn or transmitted out to the probe in proportion to the degree of impedance mismatch at the coupling interface. In a preferred embodiment, the coupling interface is configured in a manner so as to reflect most of the energy back into the horn. The horn therefore, essentially acts as an energy storage device or “reservoir”, thereby allowing a substantial increase in drive amplitude.

Since the energy coupled into the elongated probe is a small portion of the energy reflected back to the horn, changes in the transverse oscillation on the probe due to bending or damping have minimal effect on the longitudinal resonance of the horn. By decoupling the transverse probe oscillation from the longitudinal horn resonance, the electrical source of the vibrations (piezoelectric or magnetostrictive) to compensate only for shifts in the resonant frequency of the horn (due to temperature, manufacturing variations, etc.). The drive mechanism is, therefore, independent of a vibrational motion of the probe.

The transverse vibrating elongated probe of the invention does not require its terminal end be permanently affixed in intimate contact to the horn assembly, since a “hammering” action associated with longitudinal vibration is absent. The elongated probe of the invention can therefore be coupled, and not welded, to the horn via a collet assembly that grips the probe along the cylindrical surface near its terminal end in a nonpermanent way. The collet assembly of the invention therefore, allows for quick attachment and detachment of the probe from the horn assembly and source components, thereby enabling manipulation of the elongated flexible probe into anatomically curved blood vessels without hindrance by the bulky horn and energy source components. The probe of the invention can therefore be inserted into a venal cavity, positioned near the occlusion site prior to coupling it to the horn source assembly. The device is then activated to effect tissue ablation and removal, after which the probe is decoupled from the horn and source component for its easy removal from the cavity.

In one embodiment a longitudinal horn is coupled to an elongated wire catheter through a collet assembly that is rapidly attachable and detachable. In a preferred embodiment, the collet assembly comprises a quick attachment-detachment (QAD) collet assembly. The attachment point of the collet assembly to the elongated probe is located at an anti-node of the horn and the dimensions are scaled (i.e., the collet head has a relatively larger diameter at the attachment point than the diameter of the probe) to produce an optimal impedance mismatch.

The QAD collet assembly of the present invention comprises a compression clamp that is housed within an externally mounted compression housing that is capable of exerting a compressive force circumferentially along a line-contact on the compression clamp upon engagement after insertion of the proximal end of an ultrasonic probe into the compression clamp. This, in turn, causes the compression clamp to exert a compressive force on the inserted ultrasonic probe end, thereby causing the probe to be non-removably, yet releasably attached to the collet assembly. The compression clamp applies a restraining inwardly compressive force on the probe that minimizes torquing or twisting of the probe. As a result, the probe can be subject to a multiple attachment and detachment procedures, without causing probe destruction, thereby enabling its extended reuse in surgical procedures.

The coupling between the elongated probe and the horn is adjusted so as to present a relatively large impedance mismatch, and be located at an anti-node of the horn. Longitudinal waves impinging on the coupling interface are either reflected back into the horn or transmitted out to the probe in proportion to the degree of impedance mismatch at the coupling interface. In a preferred embodiment, the coupling interface is configured in a manner so as to reflect most of the energy back into the horn. The horn therefore, essentially acts as an energy storage device or “reservoir”, thereby allowing a substantial increase in drive amplitude.

The collet assembly of the present invention comprises a base segment that is capable of coupling to a compression housing segment that is removably attached to the device handle by mechanical assembly, such as for example, a screw thread comprising a locking nut, bayonet mount, keyless chuck and cam fittings. Alternatively, the rear segment of the mechanical assembly is a hollow cylindrical segment comprising a screw thread that allows insertion and attachment of the ultrasonic device handle containing a drive assembly containing a complimentary thread arrangement to be inserted into and non-removably attached to the cylindrical segment by applying a torque. In another embodiment, the ultrasonic probe is mounted to the collet assembly such that the collet assembly grips the probe at a point greater than about 1 mm and less than about 30 mm from the terminus of the probe proximal end, or optionally, is adjustable to any point in between, so as to optimize probe vibration based on the frequency of the ultrasound transducer in the device handle. In another embodiment, the probe attachment, comprising the external collet assembly with the attached probe, is connected to the operating handle of the ultrasonic device.

The elongated ultrasonic probe that is removably restrained by the collet assembly of the present invention is either a single diameter wire with a uniform cross section offering flexural stiffness along the entire length, or is tapered or stepped along its length to control the amplitude of the transverse wave along the entire longitudinal axis. Alternatively, the probe can be cross-sectionally non-cylindrical that is capable of providing both flexural stiffness and support energy conversion along the entire length. The length of the elongated probe of the present invention is chosen so as to be resonant in either in an exclusively transverse mode, or be resonant in combination of transverse and longitudinal modes to provide a wider operating range. In a preferred embodiment, the elongated probe of the present invention is chosen to be from about 30 cm to about 300 cm in length. In a most preferred embodiment, the elongated probe of the invention has a length of about 70 cm to about 210 cm. Suitable probe materials include metallic materials and metallic alloys suited for ultrasound energy transmission. In a preferred embodiment, the metallic material comprising the elongated probe is titanium.

In another embodiment, the elongated probe of the invention is circumferentially enclosed in a sheath that provides a conduit for irrigation fluids, aspiration of fragmented tissue, or for delivery of therapeutic drugs to the occlusion site. The sheath can extend either partially or over the entirety of the probe, and can additionally comprise of fenestrations for directing ultrasonic energy from the probe at specific locations within venal cavities for selective ablation of tissue. An ultrasonic tissue ablation device comprising a sheath for removal of occlusions in blood vessels has been disclosed in assignee's co-pending patent application Ser. No. 09/776,015, the entirety of which is hereby incorporated by reference

In another embodiment, the elongated catheter probe is comprised of a proximal end and a distal end with respect to the horn assembly, and is in the form of a long small diameter wire incorporating a series of telescoping segments along its longitudinal axis, such that the largest diameter segment is proximal to the horn assembly, and either continually or segmental, sequentially decreasing diameters from the proximal end to the distal end. With reference to the probe, coupling and horn assemblies as referred to in the figures describing the present invention, the proximal end for each component refers to the end farthest from the probe tip, while distal end refers to the end closest to the probe tip. In another embodiment, the elongated probe is comprised of a non-segmented, uniformly narrow diameter wire, such as for example a guide wire, such as those used in insertion of catheters.

In a preferred embodiment, the QAD collet of the invention is housed within an externally mounted compression clamp or collet assembly comprising a base segment with a longitudinal slit capable of accommodating a narrow-diameter catheter wire probe, and a compression housing that is capable of exerting a compressive force on the base after insertion of the ultrasonic probe into the longitudinal slit, thereby causing a non-removable probe attachment (“attached mode”) to the collet assembly. The collet assembly applies a restraining inwardly compressive force on the probe that minimizes torquing or twisting of the probe. As a result, the probe can be subject to a multiple attachment and detachment procedures, without causing probe destruction, thereby enabling its extended reuse in surgical procedures.

The terminal ends of the compression clamp and compression housing components of the collet assembly of the present invention are tapered so as to allow the collet assembly to maintain a true axial orientation, thereby enabling multiple insertions and retractions of the probe into and from the collet assembly prior to and after device use, without causing the probe to kink. Additionally, the shape of the proximal end of the compression clamp (rear segment with respect to the entering probe), is matched with that of the ultrasound energy source generator so as to maximize contact area between the collet assembly and the distal end of the transducer-sound conductor assembly (the “drive assembly”) The proximal end of the collet assembly is shaped in any suitable form providing maximal contact area, including conical, frusto-conical, triangular, square, oblong, and ovoid, upon probe attachment to the collet within the housing assembly, which in turn maintains intimate contact with the drive assembly. The three segment assembly that includes the probe, the collet assembly and the drive assembly, form a single assembled component in the device operational state, in terms of their combined ability to transmit sound energy from the transducer in the drive assembly to the probe without energy loss thermally or mechanically. The collet assembly of the present invention can be designed to accommodate a series of probe diameters, or for a specific probe diameter by varying the inner diameter of the cylindrical slot. The outer diameters of the collet assembly, however, remains unchanged, thereby allowing attachment of probes of differing diameters into a universal coupling and drive assembly.

The collet assembly of the present invention enables (1) attachment of the ultrasonic wire probe of the device in a rapidly detachable manner to the hand piece that either functions as a conduit for ultrasonic energy that is obtained from an externally located element (optionally, the device handle can house the ultrasonic energy source) and (2) transmission of ultrasonic energy from the source element to the ultrasonic wire probe, causing it to vibrate in a substantially transverse mode.

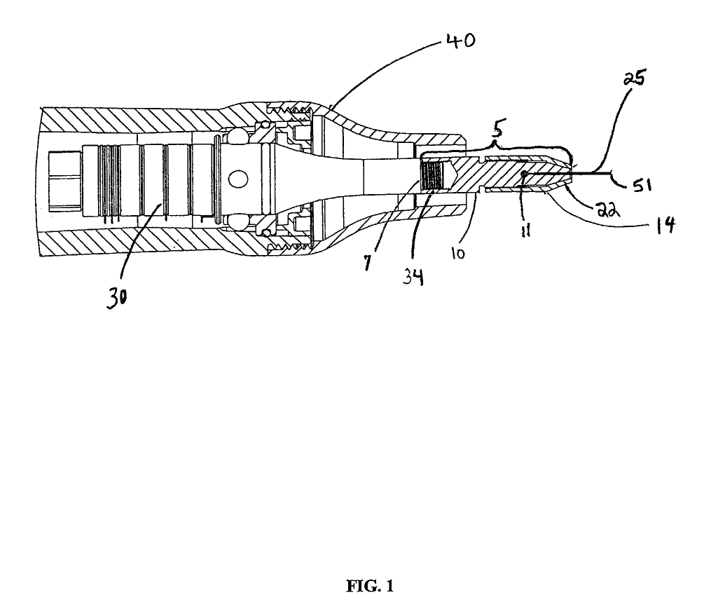

A preferred embodiment of the collet assembly of the present invention comprising a removably attached ultrasonic wire probe and a device handle comprising an ultrasonic energy source housed within is shown in FIG. 1. The collet assembly 5 comprises a compression clamp 10 having a proximal end 7 and a tapered distal end 8. The distal end 8 is removably attached to an ultrasonic wire probe 25 whereby the compression clamp 10 is made to remain in intimate contact with the probe 25 by a compression housing 14. The compression clamp 10 of the collet assembly is, in turn, removably attached to an ultrasonic energy source 30 that is housed inside a handle 40 by a thread assembly 34 in a manner so as to remain in intimate contact with the energy source 30. The collet assembly 5, therefore, maintains the ultrasonic wire probe 25 to be in contact with the ultrasonic energy source 30 indirectly, and conductively transfers ultrasonic energy from the energy source 30 to the ultrasonic wire probe 25, thereby causing the ultrasonic wire probe 25 to vibrate substantially in a transverse mode.

The collet assembly 5 of the present invention when coupled to both the ultrasonic wire probe 25 and the handle 40 housing the ultrasonic energy source 30, enables the ultrasonic wire probe 25 and the handle 40 to function as a single rigidly connected unit for efficient transfer of ultrasonic acoustic energy. The efficiency of this energy transfer is substantially influenced by the force with which the collet assembly 5 grips the ultrasonic wire probe 25 in the “attached mode” wherein the probe 25 is non-removably restrained by the collet assembly 5 which causes the probe 25 to physically remain attached to the hand piece segment of the device.

A low grip force exerted by the collet assembly 5 on the ultrasonic wire probe 25 in the attached mode results in substantial loss of energy between the collet assembly 5 and the wire probe 25. Above a threshold level, any further increase in the grip force does not increase the efficiency of energy transfer. In the embodiment shown in FIG. 1, the collet assembly grip force is maintained at an optimal level by tightening compression housing 14 over the compression clamp 10 with a calibrated torque wrench.

The grip force exerted by collet assembly 5 of the present invention on the ultrasonic wire probe 25 (in the attached mode) provides minimal surface contact between the compression clamp 10 and the compression housing 14. By maintaining such a minimal contact, the collet assembly 5 of the present invention overcomes the difficulty with regard to matching of the tapering angle at the distal end of the compression housing 14 with that of the corresponding tapered distal end of the compression clamp 10, as is the case in prior art collets.

In prior art collets for ultrasonic probes, the inner surface of the collet housing mates with the tapered surface of the collet base in an unpredictable and inconsistent manner because of the difficulty in matching the angle of taper on the housing and the base. Even though the collets can be produced on highly accurate machines (i.e., computer controlled lathes, numerically controlled screw machines, etc.), the machine tolerances still create collets that vary within a few thousandths of an inch, which is not an unusual tolerance. Thus, the position of the line contact is inconsistent from collet to collet and can only be located at either the front end or the back end of the collet and cannot be located in the middle. For the same tightening torque, the actual closing force can vary over a wide range from collet to collet.

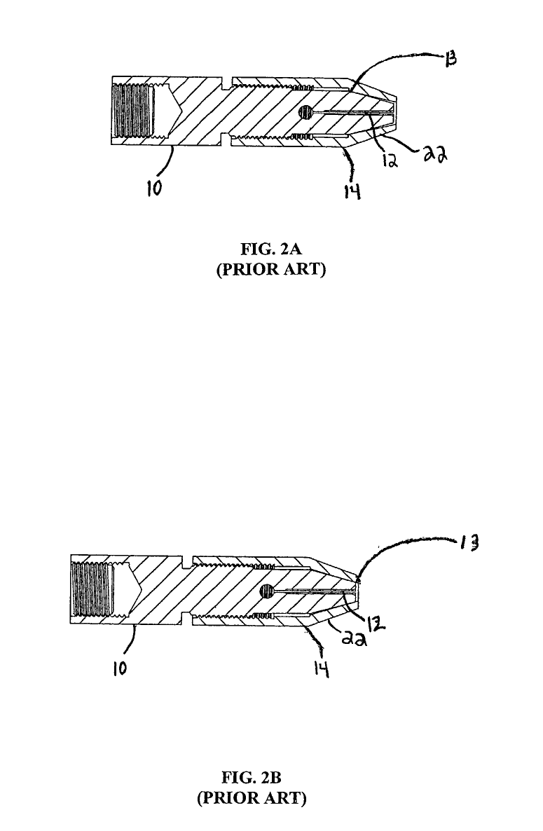

In prior art collets, a mating surface of the compression housing and a mating surface of the compression clamp mate in one of three scenarios: (1) perfect mating; (2) unevenly matched mating toward the back end of the compression housing (FIG. 2A); or (3) unevenly matched mating toward the front end of the compression housing (FIG. 2B). Each of these three mating scenarios will be discussed below. Even under perfect mating between the mating surface of the compression housing and the mating surface of the compression clamp, the friction between the two mating surfaces is substantially high because the circular slot acts as a fulcrum for closing of the longitudinal slot that accommodates the ultrasonic probe. Thus, for the same tightening torque, the actual closing force, and therefore the grip force exerted by the collet assembly on the ultrasonic wire probe in the attached mode, can vary over a wide range for one collet with respect to another. This, in turn, leads to substantial variation in the efficiency of ultrasound energy transfer from the collet assembly to the ultrasonic probe, which can seriously impact probe operation efficiency.

FIG. 2A and FIG. 2B illustrate the practical situation of random variability in the line contact between the compression housing 14 and the compression clamp 10. In the case of unevenly matched taper angles between the distal end 22 of the compression housing 14 and the distal end 8 of the compression clamp 10, a line contact and not a complete mating of the entire mating surfaces occurs between distal ends of the compression housing 14 and the compression clamp 10. Such a line contact, however, varies positionally in a random manner and results in large variations in the grip force exerted by the collet assembly 5 on the ultrasonic wire probe 25 inserted in longitudinal slot 12, thereby impacting the transfer of ultrasound energy by collet assembly 5 from the energy source 30 to the ultrasonic wire probe 25. FIG. 2A illustrates an unevenly matched mating contact 13 toward the back end of the compression housing while FIG. 2B illustrates an unevenly matched contact 13 mating toward the front end of the compression housing.

The collet assembly 5 of the present invention takes into consideration that will be significant variation in the machining, and the collet assembly 5 focuses the gripping force only on a line contact. The collet assembly 5 of the present invention overcomes the random variability of the line contact due to the relative taper angles of the compression housing 14 and the compression clamp 10 by providing a housing assembly that is designed to make a line contact with the base at a pre-determined, optimal location. The location of the line contact in the collet assembly 5 of the present invention can be controlled within a close tolerance that is consistent with the choice of machining operation for the individual components forming the collet assembly 5. Since a surface-to-surface contact between the compression housing 14 and the compression clamp 10 is eliminated, the friction between the compression housing 14 and the compression clamp 10 is minimal and a substantial proportion of the tightening torque is directed towards closing the longitudinal slot in the compression clamp 10 (i.e., collet jaw compression). The collet assembly 5 of the present invention provides a consistent grip force on the ultrasonic wire probe 25 for any selected tightening torque. A consistent grip force is exerted by the collet assembly 5 of the present invention on the ultrasonic wire probe 25 in the attached mode that in turn results in a highly efficient transfer of ultrasound energy from the collet assembly 5 to the probe 25 for optimal probe performance upon activation of the ultrasound tissue ablation device.

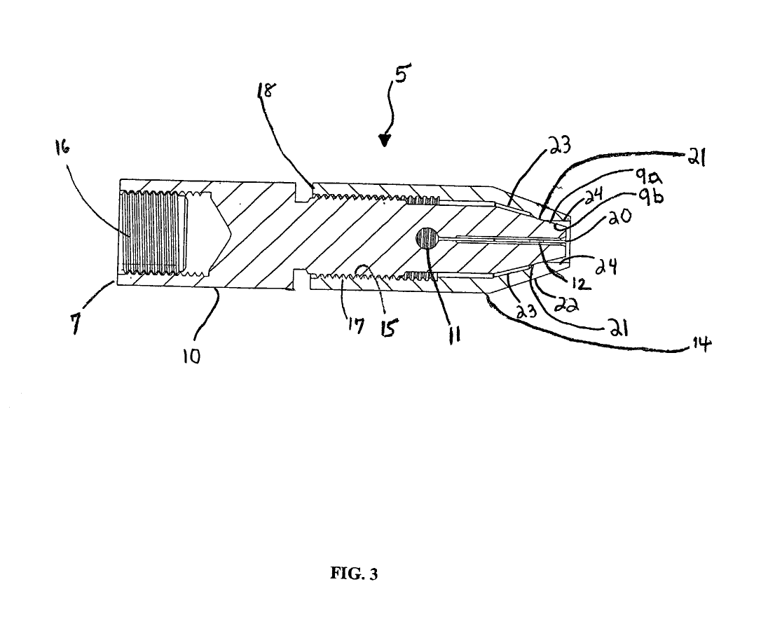

FIG. 3 shows a cross-sectional view of a preferred embodiment of the collet assembly 5 of the present invention wherein a pre-determined line-contact is established between the compression clamp 10 and the compression housing 14. As seen in FIG. 3, the collet assembly 5 comprises a cylindrical compression clamp 10 having a proximal end 7 provided with a coupling mechanism 16, and a conical, tapered distal mating surface 9a. The coupling mechanism 16 comprises a thread assembly 19 that is capable of engaging a complementary thread assembly suitably located on a horn assembly (not shown) that forms part of the ultrasonic energy source 30 of the ultrasonic tissue ablation device. The compression clamp 10 further comprises a slit 20 having a centrally located longitudinal slot 12 that extends from the distal end 8 along its longitudinal axis, terminating at a circular slot 11 extending across the diameter of the compression clamp 10, and in a direction perpendicular to the slit 20. The compression clamp 10 further comprises of a thread assembly 15 that is capable of engaging a complementary thread assembly 17 of the compression housing 14. The compression housing 14 comprises a hollow cylinder with a proximal end 18 and a tapered distal end 22. The dimensions of the compression housing 14 are chosen so as to enable it to at least partially accommodate the compression clamp 10. An inner surface of the compression housing 14 comprises a line-contact lip 21 proximal to the distal end 22, extending circumferentially along the inner surface of the compression housing 14. The line-contact lip 21 is capable of providing a mechanism for exerting a circumferential line-contact along the correspondingly located surface proximal to the distal end 8 of the compression clamp 10. In one embodiment, the line-contact lip 21 extends continuously along the inner surface of the compression housing 14 proximal to the tapered distal end 22 upon engaging the compression clamp 10 with the compression housing 14. In another embodiment, the line-contact lip 21 comprises a plurality of discontinuous arctuate segments that extend circumferentially along the inner surface of the compression housing 14 proximal to the tapered distal end 22 that are capable of providing a series of discontinuous line (or point) contacts along the correspondingly located surface proximal to the tapered distal end 8 of the compression clamp 10 upon engaging the compression clamp 10 with the compression housing 14.

The line-contact lip 21 is a surface that extends from the inner surface of the compression housing 14. In a preferred embodiment of the present invention, the line-contact lip 21 is a round surface (i.e., a dimple). Because the line-contact lip 21 is a round surface mating with a flat surface of the compression clamp 10, the line-contact lip 21 makes consistent and repeatable contact with the compression clamp 10 at a predetermined location. The round surface of the line-contact lip 21 ensures that the line-contact lip 21 mates in a continuous line all the way around the compression clamp 10, thus creating a line contact. Also, the round surface of the line-contact lip 21 ensures that the line-contact lip 21 mates in the same location every time. Those skilled in the art will recognize that the line-contact lip 21 could be other shapes within the spirit and scope of the invention.

The line-contact collet assembly of the present invention ensures consistent and repeatable contact between the compression clamp 10 and the compression housing 14 at a pre-determined location. The design of the inner surface of the compression housing 14 ensures that the line-contact lip 21 mates with the compression clamp 10 in a uniform manner. As best shown in FIG. 3, the inner surface of the compression housing 14 has a groove 23 and a notch 24. The groove 23 in the inner surface of the compression housing 14 eliminates surface to surface contact between the compression housing 14 and the compression clamp 10 toward the back end of the mating surfaces 9a and 9b. In a preferred embodiment of the present invention, the groove 23 is machined into the inner surface of the compression housing 14, although those skilled in the art will recognize the that groove 23 can be fabricated using other methods known in the art. The length and depth of the groove 23 can be varied depending on the length of line contact that is desired. Similarly, the notch 24 eliminates surface to surface contact between the compression housing 14 and the compression clamp 10 toward the front end of the mating surfaces 9a and 9b. In a preferred embodiment of the present invention, the notch 24 is machined into the inner surface of the compression housing 14, although those skilled in the art will recognize the that notch 24 can be fabricated using other methods known in the art. The length and depth of the notch 24 can be varied depending on the length of line contact that is desired. Together the groove 23 and the notch 24 ensure that the line-contact lip 21 of the compression housing 14 has consistent and repeatable contact with the compression clamp 10.

The length of the line-contact lip 21 can be varied depending on the desired length of contact between the compression housing 14 and the compression clamp 10. The location of the line-contact lip 21 is controlled in the machining process which is simple to control. On the other hand, the prior art requires controlling the taper angle of two different surfaces (the compression clamp 10 with the compression housing 14) which requires precise machining and accurate alignment and is much more difficult.

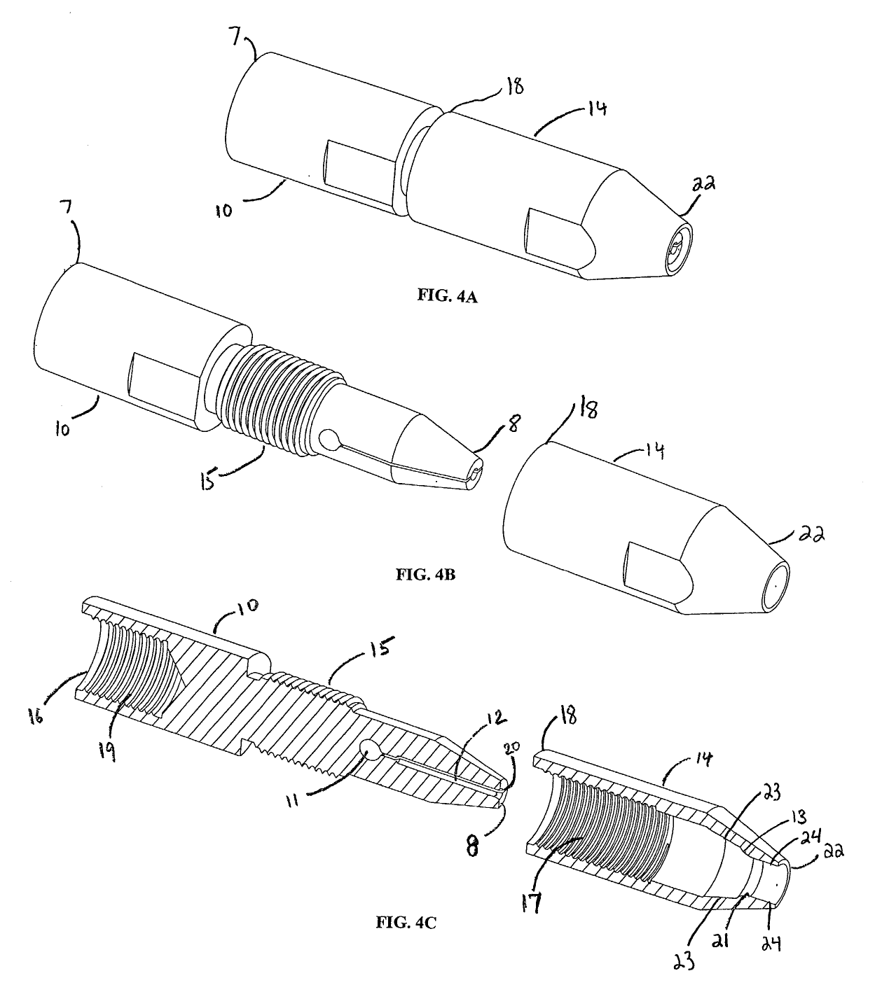

FIG. 4A and FIG. 4B show assembled and disassembled views of the collet assembly 5 of the present invention, wherein the compression housing 14 is either removably attached to the compression clamp 10 (FIG. 4A) or detached from the compression clamp 10 (FIG. 4B) by engagement and disengagement of the thread assembly 15 located along the outer surface of the compression clamp 10 with a complementary thread assembly located along the inner surface of compression housing 14 (not shown). FIG. 4C shows a cross sectional view of the collet assembly 5 of the present invention comprising the compression clamp 10 and the compression housing 14. A proximal end 7 of compression clamp 10 comprises a thread assembly 19 that is capable of engaging with complementary threading of a horn assembly (not shown) that forms part of the ultrasonic energy source 30. The compression clamp 10 further comprises a slit 20 centrally located along a longitudinal slot 12, and extends inwardly from the tapered distal end 8 along the longitudinal axis of the cylindrical compression clamp 10. The longitudinal slot 12 is capable of removably receiving the ultrasonic wire probe 25. The slit 20 terminates at a perpendicular circular slot 11 which acts as a fulcrum about which the slit 20 and consequently the circular slot 11 are compressed after receiving the probe 25 by the line-contact lip 21 of the compression housing 14 upon engaging thread assembly 15 of the compression clamp 10 with the complementary thread assembly 17 of the compression housing 14.

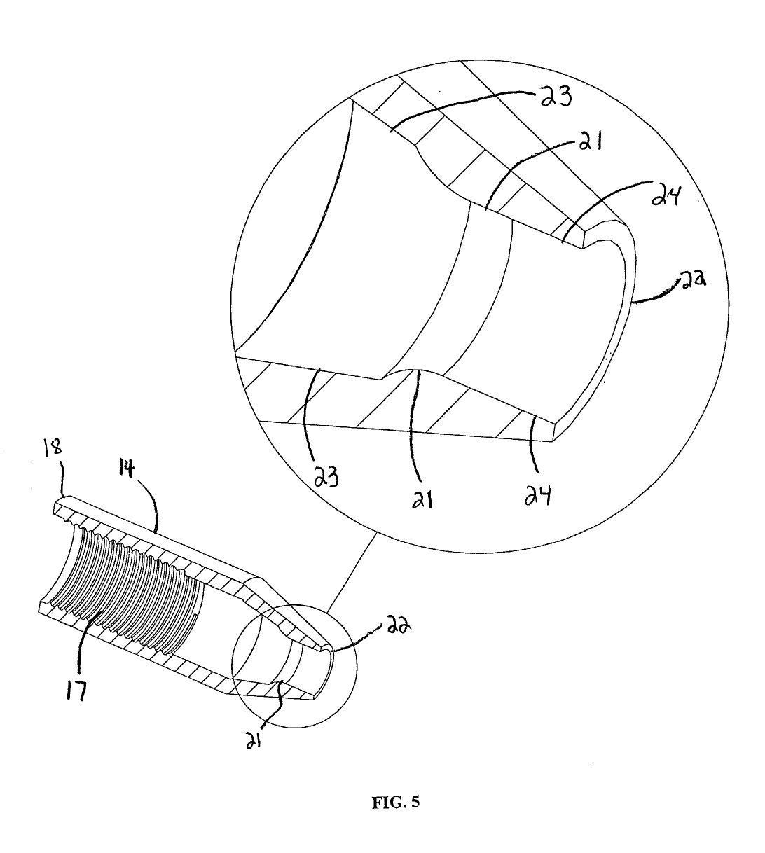

FIG. 5 shows a cross-sectional view of the compression housing 14, including an expanded view of the line-contact lip 21 that extends cirfumferentially along the inner surface of the tapered distal end 22 of the compression housing 14.

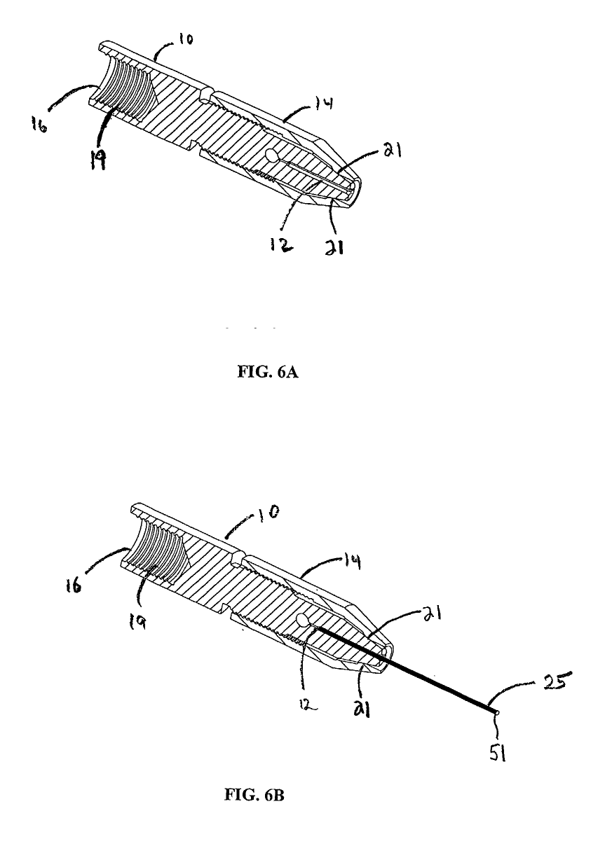

FIG. 6A and FIG. 6B show cross-sectional views of the line-contact collet assembly 5 of the present invention prior to and after attachment of an ultrasonic wire probe 25 of the ultrasonic tissue ablation device. As seen in FIG. 6A, the line-contact lip 21 in the compression housing 14 remains in intimate surface contact with the corresponding area along the circumference of the compression clamp 10 in the engaged mode. As seen in FIG. 6B, the longitudinal slot 12 is capable of removably receiving the probe 25 when the compression housing remains coupled, but not fully engaged (tightened). Following insertion of the probe 25, the compression housing 14 is tightened by applying a pre-determined torque force supplied by a mechanical device, such as for example, a calibrated torquing wrench, that results in the line-contact lip 21 exerting a uniform, compressive force circumferentially on the compression clamp 10, which in turn, causes the probe 25 to be non-removably retained within the longitudinal slot 12 in a manner so as to remain in intimate surface contact with the compression clamp 10. The proximal end 7 of the compression clamp 10 is removably attached to the horn assembly of the ultrasonic energy source (not shown) by engaging the thread assembly 19 with a complementary thread assembly in the horn assembly (not shown). Thus, in the attached mode, the collet assembly 5 of the present invention enables the ultrasonic wire probe 25 to remain in rigid, indirect contact (via the collet assembly) with the ultrasonic energy source 30 of the device that simulates a single component that results in an efficient transfer of ultrasonic energy to the source to the ultrasonic wire probe 25.

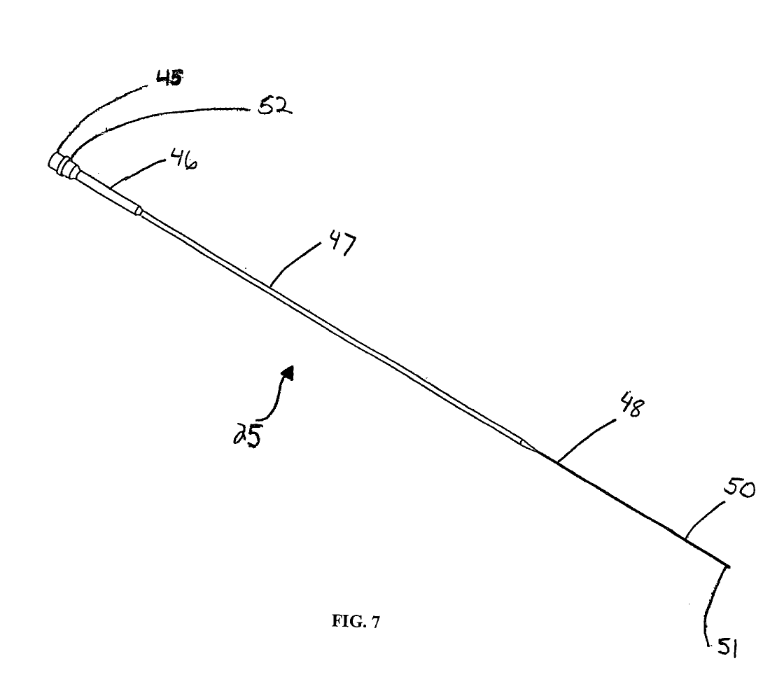

FIG. 7 shows a preferred embodiment of the elongated ultrasonic wire probe 25 of the present invention comprising a proximal end 45 and a distal end 50 that includes a probe tip 51. The probe 25 is coupled to a transducer and sound conductor assembly (not shown) that functions as generation and transmission sources of ultrasound energy for activation of the probe 25. The generation source may or may not be a physical part of the device itself. The probe 25 transmits ultrasonic energy received from the sound conductor along its length, and is capable of engaging the sound conductor component at the proximal end 45 via the collet assembly 5 with sufficient restraint to form an acoustical mass that can propagate the ultrasonic energy provided by the ultrasonic energy source (not shown). The probe diameter decreases at defined segment intervals 46, 47, and 48. The segment interval 48 which comprises the probe tip 51 at the distal end 50 is capable of flexing more than the segment intervals 46 and 47 because of the relatively smaller diameter, and thereby enables the probe 25 to generate more cavitation energy along segment interval 48 and the distal end 50. Energy from the ultrasound energy source is transmitted along the length of the probe 25, causing the probe 25 to vibrate in a direction that is transverse to the longitudinal axis of the probe 25. The segment interval 46 has a head segment 52 for engaging the collet assembly 5 of the present invention, which in turn, is attached removably to the sound conductor-transducer assembly. In a preferred embodiment of the present invention, the sound conductor component for providing, amplifying and transferring ultrasonic energy to the ultrasonic wire probe 25 is a Mason (Langevin) horn that is detachably connected to the probe 25 through the collet assembly 5.

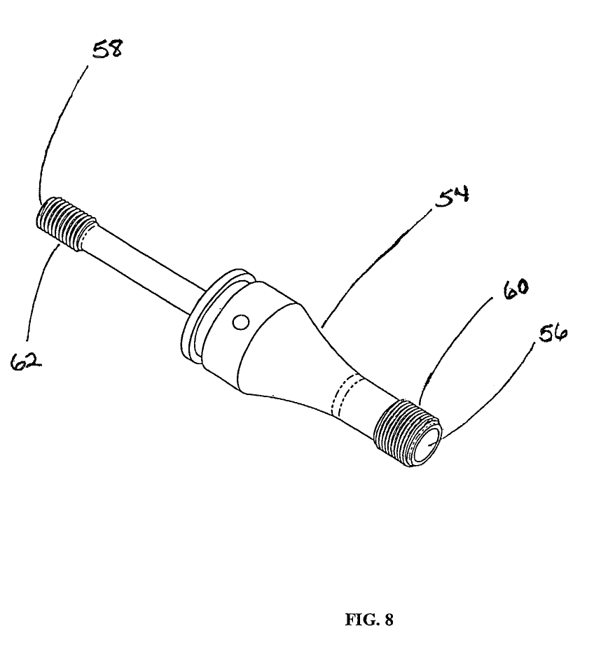

FIG. 8 shows one embodiment of the horn assembly 54 of the present invention that is detachably coupled to the proximal end 45 of the ultrasonic wire probe 25. The horn assembly 54 comprises of a distal end 56 that is capable detachably coupling to the line-contact collet assembly 5 of the present invention having removably attached thereto the ultrasonic wire probe 25 and a proximal end 58 that is coupled to a transducer (not shown) functioning as an ultrasound energy source by screw threads 60 and 62 located terminally at either end. As previously discussed, the horn assembly 54 comprising the sound conductor or “horn” functions as an energy reservoir that allows only a small fraction of the energy transmitted by the ultrasonic energy source to the probe 25 via the line-contact collet assembly 5, thereby minimizing energy loss due to probe 25 bending or damping that can occur when the probe 25 is inserted into blood vessels.

The collet assembly 5 of the present invention when used in an ultrasonic tissue ablation device provides several advantages for tissue ablation within narrow arteries over conventional devices. In the present invention, the transverse energy is transmitted extremely efficiently from the energy source (not shown) to the probe 25 by the collet assembly 5 of the present invention due to its line contact with the probe 25. The required force to cause cavitation is, therefore, low. The transverse probe vibration provides sufficient cavitation energy at a substantially low power (˜1 watt). Because transverse cavitation occurs over a significantly greater length (i.e., along the entire probe longitudinal axis that comes in contact with the tissue), the rates of endovascular materials that can be removed are both significantly greater and faster than conventional devices. The transverse vibrational mode of the elongated probe attached to the collet assembly 5 of the present invention can be attached and detached multiple times without altering the efficiency of energy transfer from the collet assembly 5 to the probe 25 due to the line contact between the compression housing 14 and the compression clamp 10 of the collet assembly 5 occurring reproducibly in a pre-determined manner.

Another advantage offered by the collet assembly 5 of the present invention is the mechanism for probe attachment and detachment by means of a lateral wall compression and decompression provided by the coupling assembly. The probe 25 can be rapidly attached to and detached from the collet assembly 5 without “screwing” or “torquing” that are utilized conventional modes of attachment of ultrasonic probes to the probe handle. This feature facilitates ease of manipulation and positioning of the probe within narrow and torturous venal cavities at the occlusion site prior to and after device use.

All references, patents, patent applications and patent publications cited herein are hereby incorporated by reference in their entireties. Variations, modifications, and other implementations of what is described herein will occur to those of ordinary skill in the art without departing from the spirit and scope of the present invention as claimed. Accordingly, the present invention is to be defined not by the preceding illustrative description but instead by the spirit and scope of the following claims.

What is claimed is:

1. An ultrasonic medical device comprising:
a probe having a proximal end and a distal end;
a collet assembly having a compression clamp and a compression housing, wherein the compression clamp engages the proximal end of the probe and the compression housing engages the compression clamp to provide a line-contact between the compression clamp and the compression housing and exert a compressive force circumferentially along the line-contact; and
a sound conductor having a proximal end and a distal end, wherein the distal end is connected to the collet assembly and the proximal end is connected to a transducer capable providing ultrasonic energy, wherein the proximal end of the probe is releasably attached to the collet assembly enabling the sound conductor to transmit the ultrasonic energy from the transducer to the collet assembly and the probe.

2. The ultrasonic device of claim 1 wherein the collet assembly detachably couples the probe to the sound conductor and the transducer to enable the probe to vibrate at an ultrasonic frequency.

3. The ultrasonic device of claim 1 wherein the compression clamp is capable of detachably engaging the compression housing to exert a compressive force on the compression clamp upon engagement of the compression housing with the compression clamp.

4. The compression housing of claim I comprising:
a hollow tube having a proximal end, a distal end, an inner surface and an outer surface; and
a conical segment proximal to the distal end of the hollow tube comprising a line-contact lip extending along at least a portion of the circumference of an inner surface that is capable of exerting a compressive force along a point of contact between the line-contact lip and the compression clamp.

5. The compression housing of claim 4 wherein the line-contact lip extends along the entire circumference along the inner surface of the conical segment.

6. The compression clamp of claim 1 comprising:
a cylindrical segment with a proximal end and a distal end;
a conical segment extending from the distal end of the cylindrical segment;
a slit extending along a longitudinal axis of the conical segment and the cylindrical segment containing a centrally located cylindrical bore capable of exerting a compressive force upon the probe inserted therein;
a mechanical assembly at the proximal end of the compression clamp capable of engaging a sound conductor; and
a mechanical assembly at the distal end of the compression clamp capable of releasably engaging the compression housing.

7. The ultrasonic device of claim 1 wherein the compression clamp is capable of releasably engaging the compression housing whereby the compression housing exerts a compressive force on the compression clamp upon engagement causing the compression clamp to engage and releasably restrain the probe inserted therein.

8. The ultrasonic device of claim 1 wherein the compression clamp transmits ultrasonic energy from the transducer to the probe when the compression clamp engages the compression housing.

9. The collet assembly of claim 1 wherein the collet assembly enables a plurality of attachment and detachment operations of the probe.

10. The ultrasonic device of claim 1 further comprising a handle having a probe attachment mechanism.

11. The ultrasonic device of claim 1 wherein the probe is a flexible guidewire.

12. The ultrasonic device of claim 1 wherein the probe further comprises a sheath assembly consisting of at least one sheath.

13. The ultrasonic device of claim 12 wherein the at least one sheath is capable of partially shielding a tissue at the site of a surgical procedure from the probe.

14. The ultrasonic device of claim 12 wherein the sheath assembly comprises an aspiration conduit, whereby fragments of occlusion materials are removed through the aspiration conduit.

15. The ultrasonic device of claim 12 wherein the sheath assembly further comprises an irrigation conduit for delivering an irrigating fluid.

16. The ultrasonic device of claim 12 wherein the sheath assembly comprises a conduit for delivering a therapeutic agent therethrough.

17. The ultrasonic device of claim 12 wherein the sheath assembly comprises an imaging system enabling positioning of the probe proximal to the occlusion.

18. The ultrasonic device of claim 12 wherein the sheath assembly is a vascular catheter comprising at least one lumen.

19. The ultrasonic device of claim 1 wherein the probe is capable of supporting a standing transverse sound waves to cause generation of ultrasonic cavitation energy in at least one location along the longitudinal axis of the probe.

20. The ultrasonic device of claim 19 wherein the ultrasonic cavitation energy is enhanced at the distal end of the probe.

21. The ultrasonic device of claim 1 wherein the diameter and flexural stiffness of the probe varies along the probe longitudinal axis.

22. The ultrasonic device of claim 1 wherein the diameter of the probe remains unchanged along the entire probe longitudinal axis.

23. The ultrasonic device of claim 1 wherein the length of the probe is between about 30 centimeters and about 300 centimeters.

24. The ultrasonic device of claim 1 wherein the length of the probe is between about 50 centimeters and about 90 centimeters.

25. The ultrasonic device of claim 1 wherein the sound conductor and the transducer are contained in a handle.

26. The ultrasonic device of claim 1 wherein the sound conductor comprises a horn assembly capable of providing an impedance mismatch between the sound conductor and the probe.

27. The ultrasonic device of claim 1 wherein the sound conductor connected to the collet assembly is capable of controlling ultrasonic energy transferred to the probe.

28. An ultrasonic device for removing occlusions in blood vessels comprising:
a wire probe having a proximal end, a distal end and a probe longitudinal axis;
a probe attachment mechanism comprising a collet assembly having a compression clamp capable of detachably engaging the wire probe and a compression housing capable of engaging the compression clamp to provide a line-contact with the compression housing and exert a compressive force circumferentially along the line-contact on the compression clamp; and
a sound conductor having a proximal end and a distal end, the distal end being connected to the collet assembly and the proximal end being connected to a transducer capable providing ultrasound energy, wherein the wire probe is releasably attached at its proximal end of the probe attachment mechanism, enabling the sound conductor to transmit ultrasound energy from the transducer to the wire probe causing the wire probe to be oscillated in a mode that is substantially transverse to the probe longitudinal axis.

29. The ultrasonic device of claim 28 wherein the collet assembly detachably couples the wire probe to the sound conductor and the transducer to enable the wire probe to vibrate at an ultrasonic frequency.

30. The ultrasonic device of claim 28 wherein the compression clamp is capable of detachably engaging the compression housing to exert a compressive force on the compression clamp upon engagement of the compression housing with the compression clamp.

31. The compression housing of claim 28 comprising:
a hollow tube having a proximal end, and a distal end, an inner surface and an outer surface; and
a conical segment proximal to the distal end of the hollow tube comprising a line-contact lip extending along at least a portion of the circumference of an inner surface that is capable of exerting a compressive force along a point of contact between the line-contact lip and the compression clamp.

32. The compression housing of claim 31 wherein the line-contact lip extends along the entire circumference along the inner surface of the conical segment.

33. The compression clamp of claim 28 comprising:
a cylindrical segment with a proximal end and a distal end;
a conical segment extending from the distal end of the cylindrical segment;
a slit extending along a longitudinal axis of the conical segment and the cylindrical segment containing a centrally located cylindrical bore capable of exerting a compressive force upon the wire probe inserted therein;
a mechanical assembly at the proximal end of the compression clamp capable of engaging a sound conductor; and
a mechanical assembly at the distal end of the compression clamp capable of releasably engaging the compression housing.

34. The ultrasonic device of claim 28 wherein the compression clamp is capable of releasably engaging the compression housing whereby the compression housing exerts a compressive force on the compression clamp upon engagement causing the compression clamp to engage and releasably restrain the wire probe inserted therein.

35. The ultrasonic device of claim 28 wherein the compression clamp transmits ultrasound energy from the transducer to the wire probe when the compression clamp engages the compression housing.

36. The collet assembly of claim 28 wherein the collet assembly enables a plurality of attachment and detachment operations of the wire probe.

37. The ultrasonic device of claim 28 further comprising a handle having a probe attachment mechanism.

38. The ultrasonic device of claim 28 wherein the wire probe is a flexible guidewire.

39. The ultrasonic device of claim 28 wherein the wire probe further comprises a sheath assembly consisting of at least one sheath.

40. The ultrasonic device of claim 39 wherein the sheath assembly is capable of partially shielding a tissue at the site of a surgical procedure from the wire probe.

41. The ultrasonic device of claim 39 wherein the sheath assembly comprises an aspiration conduit, whereby fragments of occlusion materials are removed through the aspiration conduit.

42. The ultrasonic device of claim 39 wherein the sheath assembly further comprises an irrigation conduit for delivering an irrigating fluid.

43. The ultrasonic device of claim 39 wherein the sheath assembly comprises a conduit for delivering a therapeutic agent therethrough.

44. The ultrasonic device claim 39 wherein the sheath assembly comprises an imaging system enabling positioning of the probe proximal to the occlusion.

45. The ultrasonic device of claim 39 wherein the sheath assembly is a vascular catheter comprising at least one lumen.

46. The ultrasonic device of claim 28 wherein the wire probe is capable of supporting a standing transverse sound wave to cause generation of ultrasonic cavitation energy in at least one location along the longitudinal axis of the wire probe.

47. The ultrasonic device of claim 46 wherein the ultrasonic cavitation energy is enhanced at the distal end of the wire probe.

48. The ultrasonic device of claim 28 wherein the diameter and flexural stiffness of the wire probe varies along the probe longitudinal axis.

49. The ultrasonic device of claim 28 wherein the diameter of the wire probe remains unchanged along the entire probe longitudinal axis.

50. The ultrasonic device of claim 28 wherein the length of the wire probe is between about 30 centimeters and about 300 centimeters.

51. The ultrasonic device of claim 28 wherein the length of the wire probe is between about 50 centimeters and about 90 centimeters.

52. The ultrasonic device of claim 28 wherein the sound conductor and the transducer are contained in a handle.

53. The ultrasonic device of claim 28 wherein the sound conductor comprises a horn assembly capable of providing an impedance mismatch between the sound conductor and the wire probe.

54. The ultrasonic device of claim 28 wherein the sound conductor connected to the collet assembly is capable of controlling ultrasound energy transferred to the wire probe.

55. A method of removing an occlusion in a blood vessel using an ultrasonic device comprising the following steps:
inserting a probe into the blood vessel having the occlusion;
positioning the probe at the occlusion by an axial or a rotational manipulation within the blood vessel;
attaching the probe to a collet assembly by inserting a proximal end of the probe into a compression clamp and engaging a compression housing with the compression clamp to cause a line-contact lip in the compression clamp to exert a compressive force along a line-contact on an external surface of the compression clamp whereby the compression clamp engages the probe;
activating a transducer to cause oscillation of the probe in a substantially transverse mode with respect to the longitudinal axis of the probe; and
detaching the probe from the collet assembly upon completion of surgical procedure and withdrawing the probe from the blood vessel.

56. The method of claim 55 wherein the probe is a flexible guidewire.

57. The method of claim 55 wherein the probe further comprises a sheath assembly comprising at least one sheath.

58. The method of claim 57 wherein the at least one sheath is capable of partially shielding a tissue at the site of the occlusion from the probe.

59. The method of claim 57 wherein the sheath assembly comprises an aspiration conduit, whereby fragments of occlusion materials are removed through the aspiration conduit.

60. The method of claim 57 wherein the sheath assembly further comprises an irrigation conduit enabling a supply of an irrigating fluid to the occlusion.

61. The method of claim 57 wherein the sheath assembly further comprises a conduit for delivering a therapeutic agent therethrough.

62. The method according to claims 57 wherein the sheath assembly further comprises an imaging system enabling positioning of the probe proximal to the occlusion.

63. The method according to claims 57 wherein the sheath assembly is a vascular catheter comprising at least one lumen.
